(19) United States
(12) Patent Application Publication
(10) Pub. No.: US 2024/0293011 A1
(43) Pub. Date: Sep. 5, 2024

(54) OPTICAL UNIT AND ENDOSCOPE

(71) Applicant: OLYMPUS MEDICAL SYSTEMS CORP., Tokyo (JP)

(72) Inventor: Junya HARADA, Tokyo (JP)

(73) Assignee: OLYMPUS MEDICAL SYSTEMS CORP., Tokyo (JP)

(21) Appl. No.: 18/659,320

(22) Filed: May 9, 2024

Related U.S. Application Data
(63) Continuation of application No. PCT/JP2021/041420, filed on Nov. 10, 2021.

(51) Int. Cl. A61B 1/00

(52) U.S. Cl. CPC A61B 1/00165; A61B 1/00117; A61B 1/00133; A61B 1/00158

(57) ABSTRACT

An optical unit includes: a fixed portion; a movable portion arranged movably inside the fixed portion; a first slider arranged between the fixed portion and the movable portion, the first slider being configured to slide with a movement of the movable portion; a second slider arranged between the fixed portion and the movable portion, and at a position different from a position of the first slider, the second slider being configured to slide with the movement of the movable portion; a driver that includes a coil and a magnet; and a biasing part that is arranged outside the movable portion, the biasing part being configured to cause the magnet to generate a biasing force to push the movable portion toward the fixed portion.

### CROSS-REFERENCE TO RELATED APPLICATION

This application is a continuation of International Application No. PCT/JP2021/041420, filed on Nov. 10, 2021, the entire contents of which are incorporated herein by reference.

### BACKGROUND

### 1. Technical Field

The present disclosure relates to an optical unit and an endoscope.

### 2. Related Art

In the related art, a technique that has a movable portion in which a movable lens is arranged, and that utilizes an electromagnetic actuator using a coil and a magnet, namely a voice coil motor, as a zoom function to change magnification ratios by moving the movable lens portion back and forth relative to a fixed portion and a focusing function for adjusting focus has been disclosed (for example, Japanese Patent No. 6678163).

### SUMMARY

In some embodiments, an optical unit includes: a fixed portion having a cylindrical shape; a movable portion holding a lens, the movable portion being arranged movably inside the fixed portion; a first slider that is arranged between the fixed portion and the movable portion, the first slider being configured to slide with a movement of the movable portion; a second slider that is arranged between the fixed portion and the movable portion, and at a position different from a position of the first slider, the second slider being configured to slide with the movement of the movable portion; a driver that includes a coil and a magnet, the driver being configured to move the movable portion in a direction of an optical axis of the lens relative to the fixed portion; and a biasing part that is arranged outside the movable portion, the biasing part being configured to cause the magnet to generate a biasing force to push the movable portion toward the fixed portion. The fixed portion includes a first rail portion and a second rail portion guiding a movement of the first slider and the second slider, respectively, and extending in a direction of the optical axis, the biasing part is arranged at a position facing the second rail portion through the optical axis of the lens, the first slider abuts on the first rail portion, and is pushed in a direction horizontal to the biasing force, and the second slider abuts on the second rail portion, and is pushed in a direction perpendicular to the biasing force.

In some embodiments, provided is an endoscope configured to be inserted into an inside of a subject to observe the inside of the subject. The endoscope includes: an optical unit; an imager configured to convert light guided by the optical unit into an electrical signal; and a controller configured to control driving of the optical unit. The optical unit includes: a fixed portion having a cylindrical shape; a movable portion holding a lens, the movable portion being arranged movably inside the fixed portion; a first slider that is arranged between the fixed portion and the movable portion, the first slider being configured to slide with a movement of the movable portion; a second slider that is arranged between the fixed portion and the movable portion, and at a position different from a position of the first slider, the second slider being configured to slide with the movement of the movable portion; a driver that includes a coil and a magnet, the driver being configured to move the movable portion in a direction of an optical axis of the lens relative to the fixed portion; and a biasing part that is arranged outside the movable portion, the biasing part being configured to cause the magnet to generate a biasing force to push the movable portion toward the fixed portion. The fixed portion includes a first rail portion and a second rail portion guiding a movement of the first slider and the second slider, respectively, and extending in a direction of the optical axis, the biasing part is arranged at a position facing the second rail portion through the optical axis of the lens, the first slider abuts on the first rail portion, and is pushed in a direction horizontal to the biasing force, and the second slider abuts on the second rail portion, and is pushed in a direction perpendicular to the biasing force.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a perspective view illustrating a configuration of an optical unit according to a first embodiment of the disclosure;

FIG. 2 is a exploded perspective view (Part 1) illustrating a configuration of the optical unit according to the first embodiment of the disclosure;

FIG. 3 is a exploded perspective view (Part 2) illustrating a configuration of the optical unit according to the first embodiment of the disclosure;

FIG. 4 is a plan view illustrating the configuration of the optical unit according to the first embodiment of the disclosure;

FIG. 5 is an A-A cross-section (Part 1) of the optical unit illustrated in FIG. 4;

FIG. 6 is an A-A cross section (Part 2) of the optical unit illustrated in FIG. 4;

FIG. 7 is a perspective view (Part 1) illustrating a configuration of a fixed-portion main body of the optical unit according to the first embodiment of the disclosure;

FIG. 8 is an enlarged diagram of a part of the fixed-portion main body of the optical unit according to the first embodiment of the disclosure;

FIG. 9 is a perspective view (Part 2) illustrating the configuration of the fixed-portion main body of the optical unit according to the first embodiment of the disclosure;

FIG. 10 is a perspective view illustrating a configuration of a part of the fixed-portion main body of the optical unit according to the first embodiment of the disclosure;

FIG. 11 is a perspective view (Part 1) illustrating a configuration of a movable portion of the optical unit according to the first embodiment of the disclosure;

FIG. 12 is a perspective view (Part 2) illustrating the configuration of the movable portion of the optical unit according to the first embodiment of the disclosure;

FIG. 13 is a perspective view (Part 3) illustrating the configuration of the movable portion of the optical unit according to the first embodiment of the disclosure;

FIG. 14 is a cross-section of the optical unit according to the first embodiment of the disclosure:

FIG. 15 is a diagram explaining movement of each ball and the center of gravity of the movable portion;

FIG. 16 is a diagram illustrating a configuration of a voice coil motor on a cross-section cut on a plane that passes through an axis C and that is parallel to the axis C;

FIG. 17 is a diagram for explaining a load applied on the optical unit according to the first embodiment of the disclosure;

FIG. 18 is a diagram for explaining a positional relationship between each ball and a biasing force;

FIG. 19 is a diagram for explaining manufacturing of the fixed-portion main body of the optical unit according to the first embodiment of the disclosure; and

FIG. 20 is a diagram illustrating a configuration of an endoscope system that includes an endoscope according to a second embodiment of the disclosure.

### DETAILED DESCRIPTION

### First Embodiment

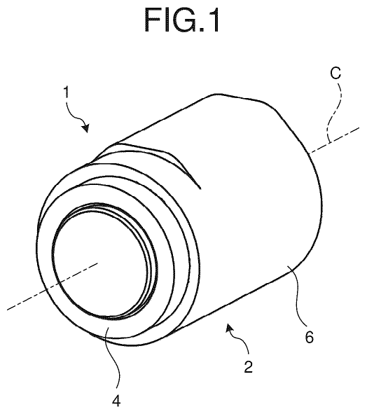

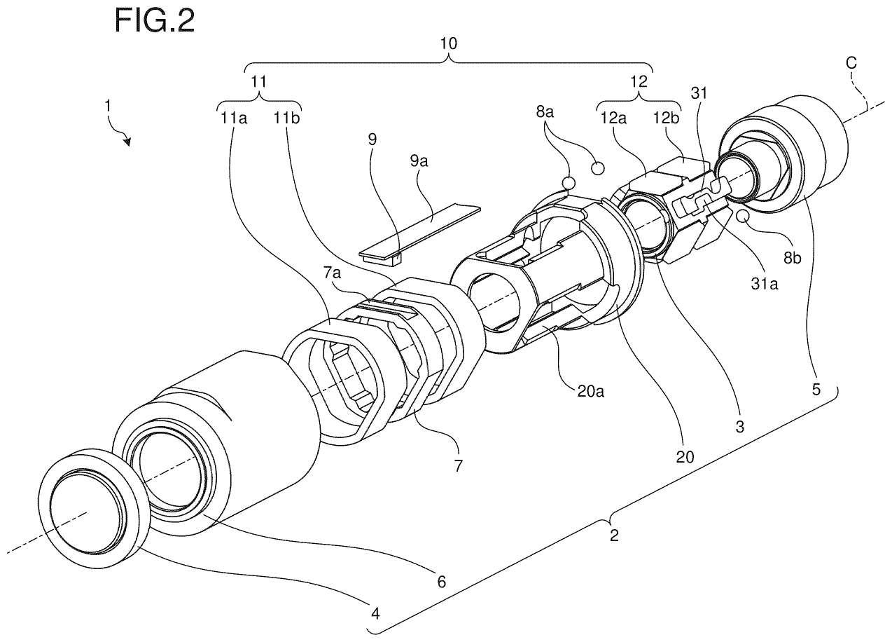

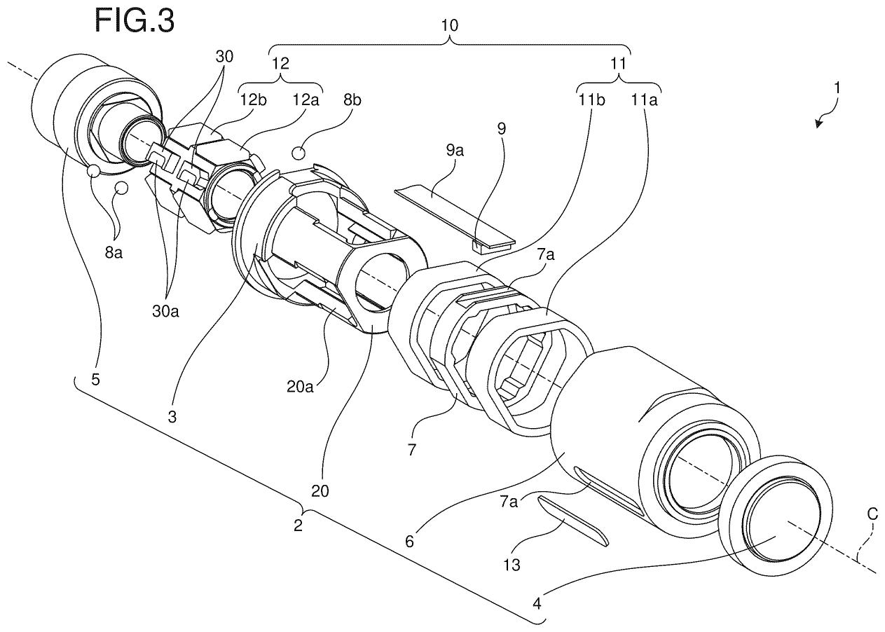

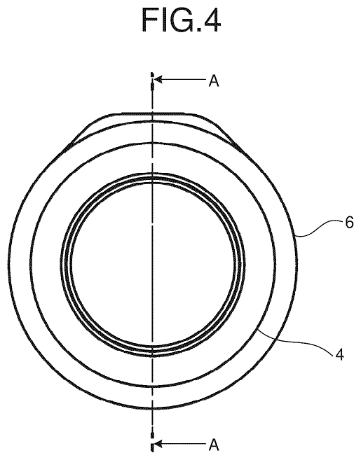

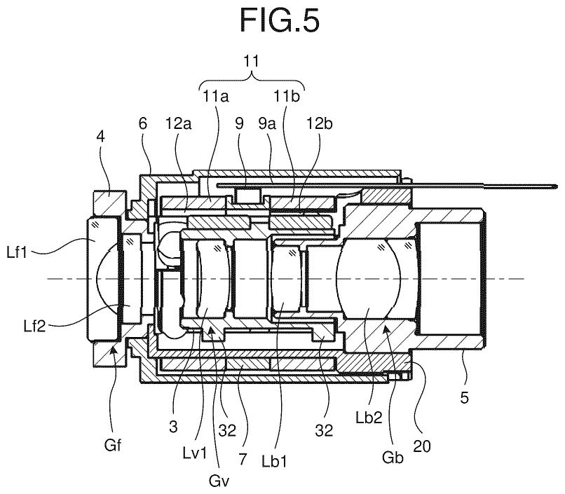

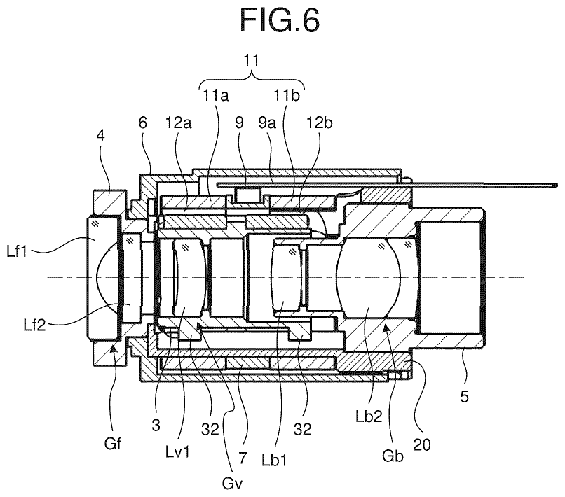

FIG. 1 is a perspective view illustrating a configuration of an optical unit according to a first embodiment of the disclosure. FIG. 2 and FIG. 3 are exploded perspective views illustrating the configuration of the optical unit according to the first embodiment of the disclosure. FIG. 4 is a plan view illustrating the configuration of the optical unit according to the first embodiment of the disclosure. FIG. 5 and FIG. 6 are A-A cross-sections of the optical unit illustrated in FIG. 4. FIG. 5 illustrates an example when a movable portion 3 moves toward an image side. FIG. 6 illustrates an example when the movable portion 3 moves toward an object side.

An optical unit 1 includes a fixed portion 2, the movable portion 3 that is movable relative to the fixed portion 2, and a voice coil motor 10 that generates a driving force to move the movable portion 3 relative to the fixed portion 2. Hereinafter, an example in which an axis C passing through the optical unit 1 coincides with an optical axis of the optical unit 1 will be explained. Hereinafter, an opposite side to the object side in a direction of the axis C is referred to as image side. In the optical unit 1 illustrated in FIG. 1, the left side is the object side, and the right side is the image side. Moreover, a center axis of each portion may also be referred to as axis C. This is because the center axis of the portion coincides with the axis C at assembling.

The fixed portion 2 includes a fixed-portion main body 20, a front frame portion 4 that is attached to the object side of the fixed-portion main body 20, and that holds an object-side fixed-lens group Gf positioned on the object side relative to a movable lens group Gv held by the movable portion 3, a rear frame portion 5 that is attached to the image side of the fixed-portion main body 20, and that holds an image-side fixed-lens group Gb positioned on the image relative to the movable lens group, and a housing portion 6 that houses the fixed-portion main body 20, the movable portion 3, and the rear frame portion 5.

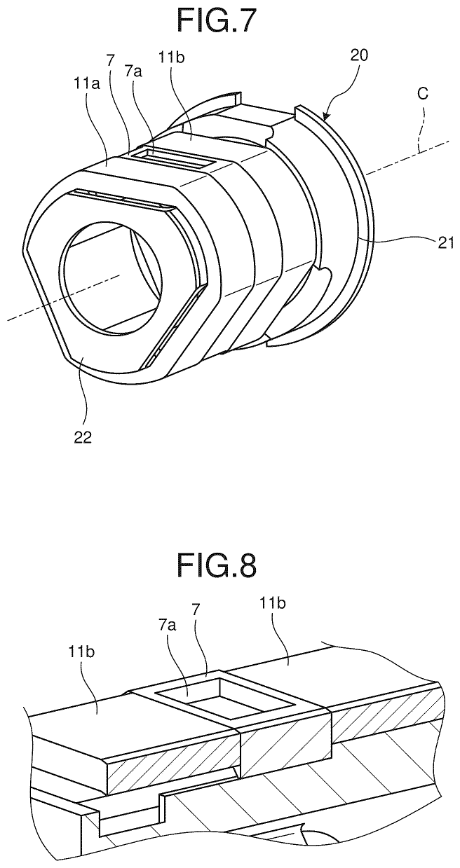

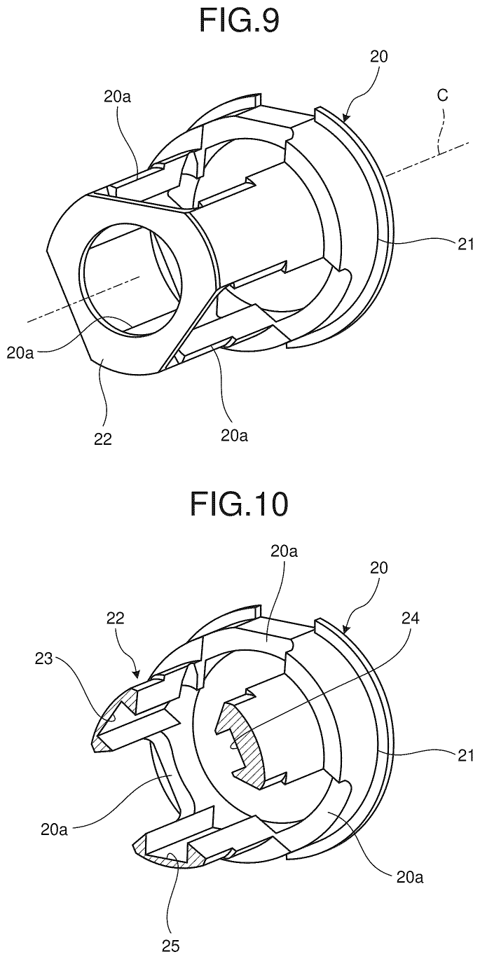

FIG. 7 is a perspective view illustrating a configuration of the fixed-portion main body of the optical unit according to the first embodiment of the disclosure. FIG. 8 is an enlarged diagram of a part of the fixed-portion main body of the optical unit according to the first embodiment of the disclosure. FIG. 9 is a perspective view illustrating a configuration of the fixed-portion main body of the optical unit according to the first embodiment of the disclosure. FIG. 10 is a perspective view illustrating a configuration of a part of the fixed-portion main body of the optical unit according to the first embodiment of the disclosure. The fixed-portion main body 20 is constituted of a cylindrical portion, and the axis C passes through the center of an interior of a cylinder. The fixed-portion main body 20 includes a first cylindrical portion 21 having the axis C as the center axis, and a second cylindrical portion 22 that extends in the direction of the axis C from one end of the first cylindrical portion 21.

The first cylindrical portion 21 has a stepped shape formed by protrusion of an end portion of an outer peripheral portion on the image side. A portion of the first cylindrical portion 21 is housed in the housing portion 6, and a stepped portion abuts on the housing portion 6.

The second cylindrical portion 22 has a cylindrical shape having an outer diameter that is smaller than an outer diameter of the first cylindrical portion 21. On a side surface of the second cylindrical portion 22, three through holes 20a that pierces through in a direction (radial direction) perpendicular to the axis C are formed (refer to FIG. 9). Moreover, on an inner peripheral surface of the second cylindrical portion 22, three rail portions (rail portions 23 to 25) that extend in the direction of the axis C are formed (refer to FIG. 10). Through holes 30a and the rail portions 23 to 25 are respectively formed at regular intervals of 120° along a circumferential direction on a cross-section in which a plane perpendicular to the axis C is the cutting plane. The through holes 30a and the rail portions 23 to 25 are arranged alternately in a circumferential direction of the second cylindrical portion 22. The rail portions 23 to 25 face the through holes 20a with respect to the axis C.

Moreover, in a center portion in the direction of the axis C of the second cylindrical portion 22, a ring-shaped portion 7 that surrounds the outer periphery of the second cylindrical portion 22 is arranged (refer to FIG. 7). In a portion of the outer periphery of the ring-shaped portion 7, a housing groove 7a having a concave shape is formed. In the housing groove 7a, a detector 9 that outputs a detection signal to detect a position of the movable portion 3 is arranged. By arranging the detector 9 in the housing groove 7a, the detector 9 is positioned between a first coil 11a and a second coil 11b described later in the direction of the axis C. The detector 9 is connected to a substrate 9a. The substrate 9a is connected to a control substrate (not illustrated) that performs position detection. The detector 9 is constituted of, for example, a magnetic detector. The magnetic detector is implemented by using, for example, a Hall element and a magnetoresistive element (MR element). The magnetic detector is fixedly arranged, housed in the housing groove 7a. Based on a detection signal detected by the magnetic detector, a position of the movable portion 3 can be accurately detected. The detection signal is related to magnetism of a magnet 12, and includes, for example, information indicating a magnetic field direction and a magnetic field intensity.

The front frame portion 4 holds the object-side fixed-lens group Gf. The object-side fixed-lens group Gf have multiple lenses (in this example, an object lens Lf1 and a lens Lf2) including the object lens Lf1 aligned in the direction of the axis C.

The rear frame portion 5 holds the image-side fixed-lens group Gb. The image-side fixed-lens group Gb have multiple lenses (lenses Lb1, Lb2) aligned in the direction of the axis C.

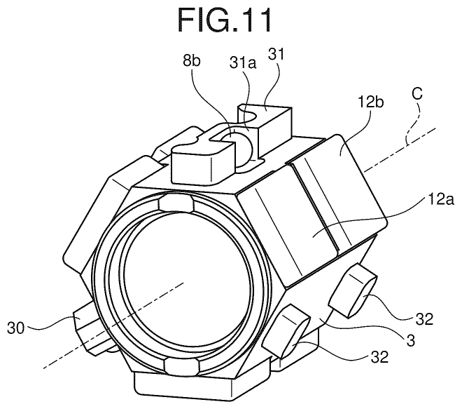

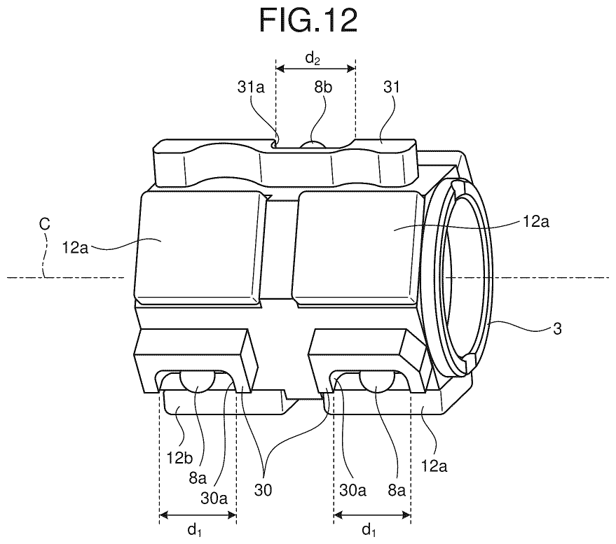

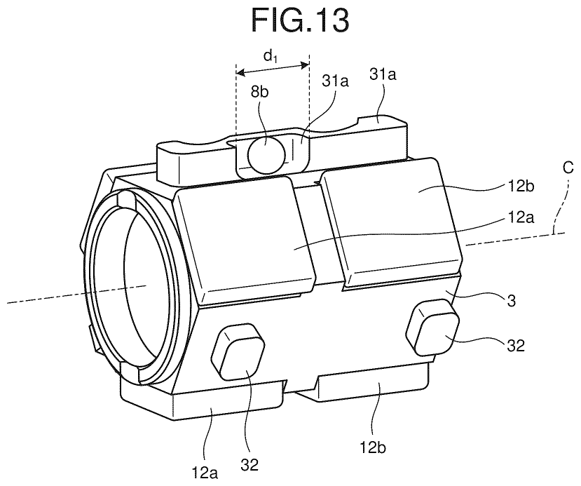

FIG. 11 to FIG. 13 are perspective view illustrating a configuration of the movable portion of the optical unit according to the first embodiment of the disclosure. The movable portion 3 is constituted of a cylindrical portion with a hexagonal-shaped outer edge when viewed from the direction of the axis C. The movable portion 3 holds the movable lens group Gv. The movable lens group Gv is constituted of a single lens or multiple lenses (in this example, a lens Lv1) aligned in the direction of the axis C.

The movable portion 3 includes a first protruding portion 30, a second protruding portion 31, and a third protruding portion 32 that are arranged on an outer surface, and that protrude outward. The first protruding portion 30, the second protruding portion 31, and the third protruding portion 32 are respectively arranged on faces that are different from one another, and that are not adjacent to one another out of six faces constituting the outer surface of the movable portion 3. That is, the first protruding portion 30, the second protruding portion 31, and the third protruding portion 32 are arranged with gaps equivalent to one face of the hexagon constituting the outer surface of the movable portion 3.

The first protruding portion 30 is housed in the rail portion 23 when it is assembled in the optical unit 1. The first protruding portion 30 has a concave portion 30a. The concave portion 30a has a groove shape in which one end in a circumferential direction of the movable portion 3 is open. The first protruding portion 30 are provided by arranging two aligned in the direction of the axis C.

The second protruding portion 31 is housed in a rail portion 24 when it is assembled in the optical unit 1. The second protruding portion 31 has a concave portion 31a. The concave portion 31a has a groove shape in which one end in the circumferential direction of the movable portion 3 is open.

A distance d₁ in the direction of the axis C of the concave portion 30a is set to be equal to or larger than a moving distance of the movable portion 3.

Moreover, a distance d₂ in the direction of the axis C of the concave portion 31a is set to be equal to or larger than the moving distance of the movable portion 3.

The distance d₁ and the distance d₂ may be identical to each other, or may be different distances from each other as long as they are set to be equal to or larger than the moving distance.

The third protruding portion 32 has a pillar shape that extends in a direction perpendicular to the axis C.

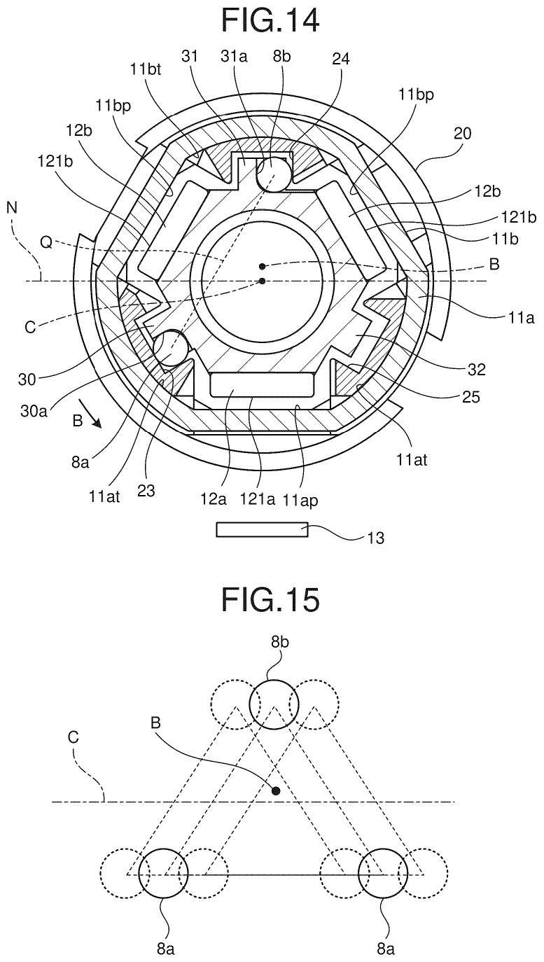

FIG. 14 is a cross-section of the optical unit according to the first embodiment of the disclosure. FIG. 14 illustrates cross-sections on different cutting planes with an axis N perpendicular to the axis C as their boundary.

Between each of the concave portions 30a and the rail portion 23, a first ball 8a is present. The first ball 8a that is housed in one of the concave portion 30a corresponds to a first slider, and the first ball 8a that is housed in the other concave portion 30a corresponds to a third slider.

Moreover, between the concave portion 31a and the rail portion 24, a second ball 8b is present. The second ball 8b housed in the concave portion 31a corresponding to a second slider.

In a state in which the movable portion 3 is housed in the fixed-portion main body 20, the respective first balls 8a are sandwiched between the second cylindrical portion 22 and the rail portion 23. In this case, one of the first ball 8a abuts on a wall surface of the rail portion 23, and the other one of the first ball 8a also abuts on a side wall of the rail portion 23.

The third protruding portion 32 is positioned in the rail portion 25 in a state in which the movable portion 3 is housed in the fixed-portion main body 20.

Subsequently, a configuration of the voice coil motor 10 will be explained. The voice coil motor 10 includes a coil 11 that is arranged in the fixed-portion main body 20 of the fixed portion 2, and a magnet 12 that is arranged in the movable portion 3 facing the coil 11 (for example, refer to FIG. 2, FIG. 3, and FIG. 14). The voice coil motor 10 functions as a driver.

The coil 11 includes a first coil 11a that is wound around an outer periphery of the second cylindrical portion 22 of the fixed-portion main body 20, and a second coil 11b that is aligned in the direction of the axis C of the first coil 11a, and that is wound on an outer periphery of the second cylindrical portion 22 of the fixed-portion main body 20 (for example, refer to FIG. 7). Between the first coil 11a and the second coil 11b, the ring-shaped portion 7 is arranged. The coils 11 may be provided by arranging pre-wound ones later. The first coil 11a and the second coil 11b adjacent to each other in the direction of the axis C are preferable to be electrically connected in series, but may be connected in parallel.

The first coil 11a and the second coil 11b include flat surface portions 11ap and 11bp, respectively, that face the through holes 20a of the fixed-portion main body 20. Moreover, the first coil 11a and the second coil 11b include cylindrical portions 11at and 11bt, respectively, that face the second cylindrical portion 22. the first coil 11a has a form in which the three flat surface portions 11ap and the three cylindrical portions 11bt are arranged alternately on a cross-section perpendicular to the axis C. Similarly, the second coil 11b has a form in which the three flat surface portions 11bp and the three cylindrical portions 11bt are arranged alternately on a cross-section perpendicular to the axis C.

The magnet 12 faces the respective flat surface portions 11ap and 11bp inside the flat surface portion 11ap of the first coil 11a and the flat surface portion 11bp of the second coil 11b, and includes three flat-shaped first magnets 12a and three flat-shaped second magnets 12b that are aligned in the direction of the axis C. The three sets of the first magnet 12a and the second magnet 12b are arranged at regular intervals of 120° along a circumferential direction on a cross-section having a plane perpendicular to the axis C as a cutting surface. By arranging the respective sets of magnets at regular intervals, the first magnets 12a and the second magnets 12b can be arranged stably. This enables to form a stable magnetic field in the voice coil motor 10, and to suppress wobbling of the movable portion 3 that moves relative to the fixed portion 2. Although the magnets 12 are arranged around the axis C at intervals of 120° each in the first embodiment, the magnets 12 may be arranged at different angular intervals.

A total of widths of the first magnet 12a and the second magnet 12b in the direction of the axis C is preferable to be shorter than a total of widths of the first coil 11a and the second coil 11b in the direction of the axis C. By satisfying this condition, within a moving range of the movable portion 3, it is possible to make the first magnet 12a and the second magnet 12b always be present within a width of the first coil 11a and the second coil 11b in the direction of the axis C.

FIG. 15 is a diagram explaining about movement of each ball and a center of gravity of the movable portion. FIG. 15 illustrates an example of arrangement of the respective balls when viewed from a direction perpendicular (for example, a direction toward the axis C from an upper left portion in FIG. 14) to the axis C and a line segment (for example, line Q in FIG. 14) connecting the center of gravity of the first ball 8a and the center of gravity of the second ball 8b, and an arrangement of each ball after movement of the movable portion 3. FIG. 15 illustrates a ball (solid line) positioned in the center of a moving range of the movable portion 3 and a ball (broken line) at positions when the movable portion 3 has moved to the image side and the object side from a central portion. The two first balls 8a and the second ball 8b are arranged at positions such that the center B of gravity of the movable portion 3 is located inside a triangle regardless of a position of the movable portion 3, in the triangle formed by connecting the centers of gravities of the respective first balls 8a and the center of gravity of the second ball 8b.

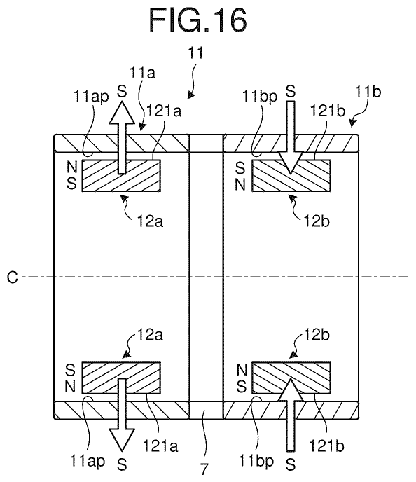

FIG. 16 is a diagram illustrating a configuration of only the voice coil motor on a cross-section that passes through the axis C and that is cut on a plane parallel to the axis C as a cutting plane. FIG. 16 illustrates different cutting planes with the axis C as their boundary. The first magnet 12a and the second magnet 12b that are paired in the direction of the axis C are arranged to be separated from each other. The set of the first magnets 12a and the set of the second magnets 12b are respectively magnetized in a radial direction, and have magnetic poles opposite to each other. In the case illustrated in FIG. 16, the first magnet 12a has its N pole on a side closer to the first coil 11a, and its S pole on the opposite side, and the second magnet 12b has its S pole on a side closer to the second coil 11b and its N pole on the opposite side. In this case, a direction of magnetic polarization of the first magnet 12a and the second magnet 12b is perpendicular to the axis C (refer to a white arrow S indicated in FIG. 16). More generally speaking, the direction of magnetic polarization of the first magnet 12a and the second magnet 12b can be any direction as long as it intersects the axis C.

In the first embodiment, the coil 11 is preferable to be wound such that a winding direction reverse between the set of the first magnets 12a and the set of the second magnets 12b. For example, when the first coil 11a is wound in a direction of an arrow B, the second coil 11b may be wound in the opposite direction. Alternatively, the first coil 11a and the second coil 11b may be connected by winding the first coil 11a and the second coil 11b in an identical direction, and by reversing the direction of the electric current. In this case, when an electric current is flowed in a direction of an arrow B indicated in FIG. 11 to the first coil 11a, the electric current is to be flowed in the opposite direction to the second coil 11b.

Moreover, in an outer portion of the housing portion 6, a housing groove 6a is formed. In the housing groove 6a, a magnetic material 13, which is a biasing part, is arranged. The magnetic material 13 attracts the magnet 12 by magnetic force, and thereby brings the movable portion 3 toward the fixed-portion main body 20. The biasing material is not limited thereto, and may be any material as long as it is capable of attracting the movable portion 3 toward the fixed-portion main body 20. For example, it may be a metal, such as iron. Furthermore, the biasing part may be configured to attract the movable portion 3 toward the fixed-portion main body 20 by applying force in a direction of separating the magnet 12 from the biasing part.

In a state in which the movable portion 3 is housed in the fixed-portion main body 20, the movable portion 3 and the fixed-portion main body 20 are not in direct contact with each other, and a relative positional relationship viewed from the direction of the axis C is such that positions of the movable portion 3 and the fixed-portion main body 20 are fixed through the two first balls 8a and the second ball 8b.

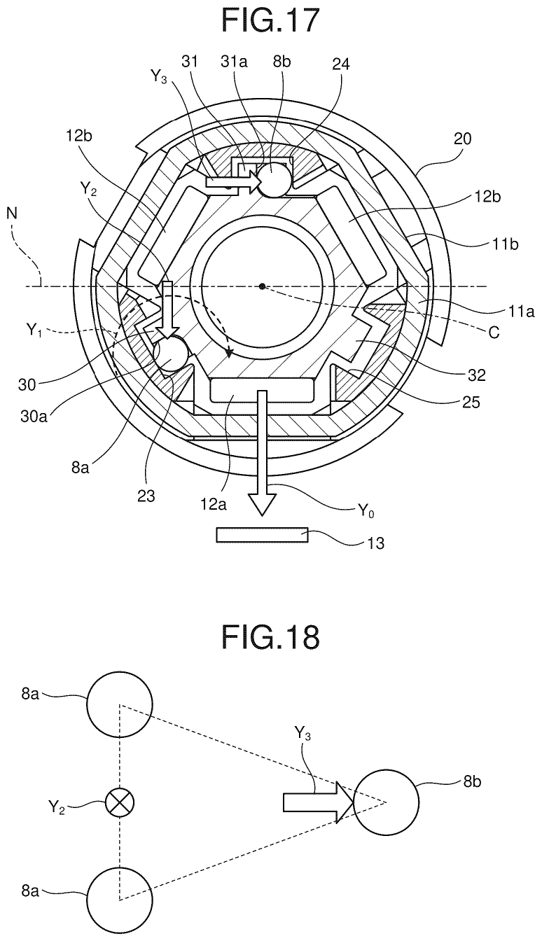

FIG. 17 is a diagram for explaining a load on the optical unit according to the first embodiment of the disclosure. FIG. 17 illustrates cross-sections having different cutting planes with the axis N perpendicular to the axis C as their boundary, similarly to FIG. 14. In the optical unit 1, the magnet 12 (the first magnet 12a in FIG. 17) receives a biasing force Y₀ by magnetic force of the magnetic material 13, to be attracted toward the magnetic material 13. By this attraction, a rotational moment Y₁ around the first ball 8a is applied to the movable portion 3. At this time, to the movable portion 3, a pressing force Y₂ with which the first protruding portion 30 pushes the first ball 8a toward the rail portion 23 is applied by the biasing force Y₀, and a pressing force Y₃ with which the second protruding portion 31 pushes the second ball 8b toward the rail portion 24 is also applied by the rotational moment Y₁.

FIG. 18 is a diagram for explaining a positional relationship between each ball and the biasing force. FIG. 18 is a diagram illustrating an example of a positional relationship among the two first balls 8a and the second ball 8b projected on a plane parallel to the axis N and passing through the axis C in the direction of Y₀. In the triangle formed by connecting the centers of gravities of the first balls 8a and the center of gravity of the second ball 8b (broken line in FIG. 18), only a component orthogonal to the biasing force Y₀ (only the pressing force Y₃) is applied to the second ball 8b. Therefore, the biasing force Y₀ is to be applied to the respective first balls 8a from a position on a line segment (broken line) between the first balls 8a. A component ratio of the biasing force Y₀ applied to the respective first balls 8a varies according to a relative position (position of the movable portion 3) of the respective balls 8a. By positioning a point of action of the biasing force Y₀ at a central position in the direction of the axis C relative to the position of the respective first balls 8a, it is possible to make the component ratio of the biasing force Y₀ applied to the respective first balls 8a uniform. By this application of the pressing force Y₂, a component in a direction of the pressing force Y₂ out of the biasing force Y₀ from the magnetic material 13 is utilized effectively, and it is possible to stabilize the position of the movable portion 3 relative to the fixed-portion main body 20.

In the optical unit 1 having the configuration described above, the movable portion 3 in which the first magnet 12a is arranged facing the first coil 11a inside in the radial direction of the fixed-portion main body 20 wound on the first coil 11a is provided. Therefore, the flat surface portion 11ap of the first coil 11a is present in a magnetic field in a direction orthogonal to a plane 121a (refer to FIG. 16) outside in the radial direction of the respective first magnets 12a. The second magnet 12b is also configured similarly. Therefore, the driving efficiency is improved, and it becomes possible to move the movable portion 3 swiftly. Moreover, by forming the surface 121a outside in the radial direction of the first magnet 12a and the surface 121b outside in the radial direction of the second magnet 12b into flat surfaces, assembly of the optical unit 1 becomes easy.

Furthermore, when an electric current is flowed through the coil 11 of the optical unit 1, a force in the direction of the axis C is generated in the movable portion 3 by an influence of a magnetic field of the magnet 12, and the movable portion 3 moves in the direction of the axis C relative to the fixed portion 2. For example, by controlling the respective electric currents to be flowed through the first coil 11 and the second coil 11b, it is possible to move the movable portion 3 relative to the fixed portion 2. In this case, the first ball 8a and the second ball 8b slide (in this example, rotate) in synchronization with the movement of the movable portion 3. By rotation of the first ball 8a and the second ball 8b, friction between the fixed-portion main body 20 and the movable portion 3 is reduced. Even in a state in which the movable portion 3 moves relative to the fixed portion 2, a surface outside in the radial direction of the magnet 12 is arranged in the through hole 20a of the fixed-portion main body 20.

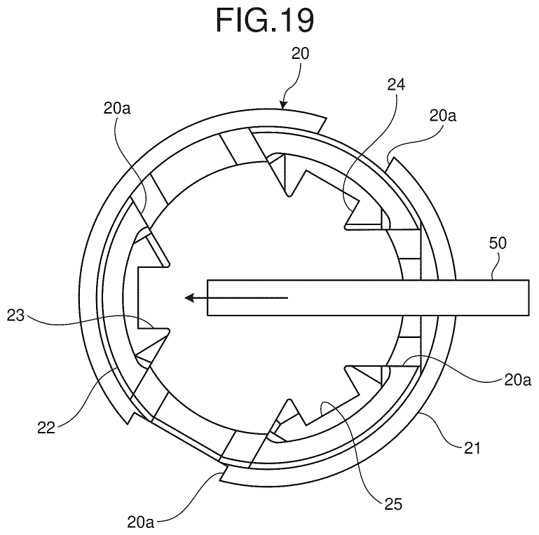

Manufacturing of the fixed-portion main body 20 will be explained, referring to FIG. 19. FIG. 19 is a diagram for explaining about the manufacturing of the fixed-portion main body of the optical unit according to the first embodiment of the disclosure. In the fixed-portion main body 20, for example, the rail portions 23 to 25 are formed by a cutter 50. In the fixed-portion main body 20, the rail portions 23 to 25 are formed to face the through holes 20a, respectively. Accordingly, by inserting the cutter 50 into the through holes 20a to form the rail portions 23 to 25, the rail portions 23 to 25 can be formed easily.

According to the first embodiment explained above, by arranging the first balls 8a and the second ball 8b between the fixed portion 2 and the movable portion 3, and by moving the movable portion 3 relative to the fixed portion 2 by rotation of the balls, the movable portion 3 (movable lens) can be moved smoothly relative to the fixed portion 2.

Moreover, according to the first embodiment, because the detector 9 is positioned between the first coil 11a and the second coil 11b in the direction of the axis C, an influence of leakage magnetic field of the coil 11 can be reduced, and reduction in detection accuracy caused by the leakage magnetic field can be suppressed. Particularly, when the detector 9 and the magnets 12 (the first magnets 12a or the second magnets 12b) are separated by movement of the movable portion 3, the influence of the leakage magnetic field becomes large, but in the first embodiment, the detector 9 is positioned in such a manner that it is less susceptible to the influence of the leakage magnetic field and, therefore, the detection accuracy can be ensured. Moreover, by arranging the detector 9 between the coils, it is possible to downsized compared to a case in which the detector 9 is positioned outward in the radial direction of the coil 11.

Furthermore, according to the first embodiment, by configuring the fixed portion 2 by using the fixed-portion main body 20, the front frame portion 4, and the rear frame portion 5, it is possible to reduce the number of parts and assembly processes, and to increase flexibility in design, thereby enabling cost reduction to be achieved.

Moreover, according to the first embodiment, because the coil 11 is wound about the axis C, it is possible to make the sliding axis of the movable portion 3 and the axis of action of a propulsive force generated by the voice coil motor 10 the same, thereby enabling stable driving.

Furthermore, according to the first embodiment, the magnets 12 are arranged in plurality in a symmetric manner with respect to the axis C. Therefore, it is possible to increase the driving force stably.

Moreover, according to the first embodiment, the magnet 12 includes multiple sets of the first magnet 12a and the second magnet 12b that are adjacent to each other along the direction of the axis C, and that have the opposite directions of magnetic polarization, the first magnets 12a have the identical direction of magnetic polarization, the coil 11 includes the first coil 11a facing the first magnets 12a, and the second coil 11b that faces the second magnets 12b and that is connected to the first coil 11a, and the directions in which an electric current flows in the first coil 11a and the second coil 11b are reverse and, therefore, the driving force can be increased.

### Second Embodiment

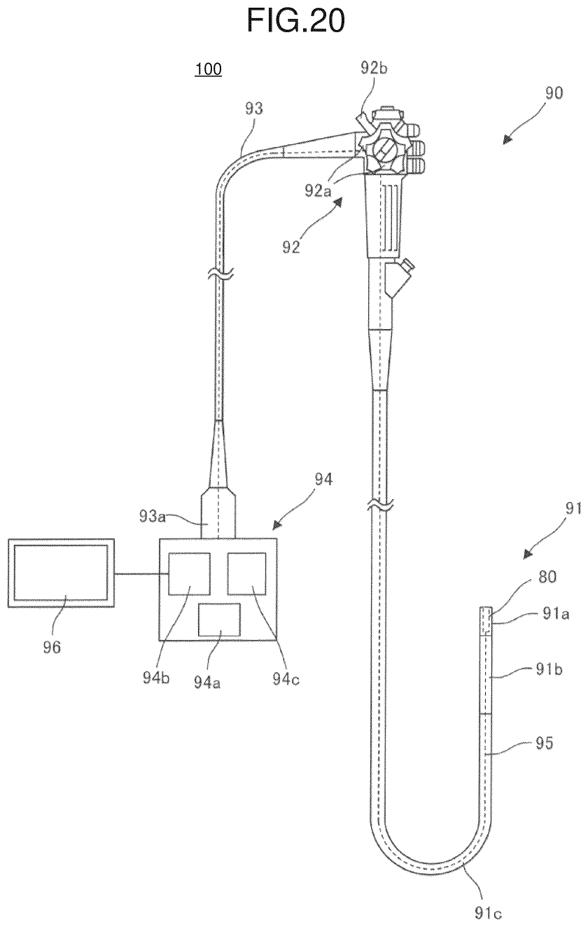

FIG. 20 is a diagram illustrating a configuration of an endoscope system that includes an endoscope according to a second embodiment of the disclosure. An endoscope system 100 illustrated in the drawing includes an endoscope 90, a control device 94, and a display device 96. The endoscope 90 includes the optical unit 1 described above. In the second embodiment, it will be explained assuming that, for example, the optical unit 1 according to the first embodiment is equipped.

The endoscope 90 can be introduced inside a body of a subject, such as human body, and optically images a predetermined observation part in the subject. The subject to which the endoscope 90 is introduced is not limited to a human body, and may also be other living bodies, or may be an artificial object, such as a machine and a building structure. In other words, the endoscope 90 may be a medical endoscope or an industrial endoscope.

The endoscope 90 includes an insertion portion 91 configured to be inserted into the inside of a subject, an operating portion 92 that is positioned at a proximal end of the insertion portion 91, and a universal cord 93 as a composite cable that extends from the operating portion 92.

The insertion portion 91 includes a distal end portion 91a arranged at a distal end, a bendable portion 91b that is bendable and arranged on a proximal end side of the distal end portion 91a, and a flexible tubular portion 91c that is arranged on a proximal end side of the bendable portion 91b, connected to a distal end side of the operating portion 92, and has flexibility. In the distal end portion 91a, an imaging portion 80 that gathers light from a subject to image the subject is arranged. The imaging portion 80 includes the optical unit 1 that gathers light from the subject, and an imager that subjects the light gathered by the optical unit 1 to photoelectric conversion, to output. The imager is constituted of a charge coupled device (CCD) or a complementary metal oxide semiconductor (CMOS). The endoscope 90 may be a rigid endoscope that does not include the flexible tubular portion 91c in the insertion portion 91.

The operating portion 92 includes an angle operating portion 92a that operates a bending state of the bendable portion 91b, and a zoom operating portion 92b that instructs the operation of the voice coil motor 10 described above, and performs zooming operation in the optical unit 1. The angle operating portion 92a is formed in a knob form, and the zoom operating portion 92b is formed in a lever form, but the respective parts may take other forms, such as a volume switch and a push switch.

The universal cord 93 is a part that connects the operating portion 92 and the control device 94. The endoscope 90 is connected to the control device 94 through a connector 93a arranged at a proximal end portion of the universal cord 93.

To the insertion portion 91, the operating portion 92, and the universal cord 93, a cable 95, such as a wire, an electric line, and a fiber, is inserted through.

The control device 94 includes a drive controller 94a that controls a bending state of the bendable portion 91b, an image controller 94b that controls the imaging portion 80, and a light-source controller 94c that controls a light source device not illustrated. The control device 94 includes a processor, such as a central processing unit (CPU), and comprehensively controls the entire endoscope system 100.

The drive controller 94a includes an actuator, and is mechanically connected to the operating portion 92 and the bendable portion 91b through a wire. The drive controller 94a controls a bending state of the bendable portion 91b by moving the wire back and forth.

The image controller 94b is electrically connected to the imaging portion 80 and the operating portion 92 through the electric line. The image controller 94b performs the drive control of the voice coil motor 10 included in the imaging portion 80, and processing of an image that captured by the imaging portion 80. The image processed by the image controller 94b is displayed on the display device 96.

The light-source controller 94c is optically connected to the light source and the operating portion 92 through an optical fiber. The light-source controller 94c controls brightness of light of the light source irradiated from the distal end portion 91a.

The operating portion 92 may be formed separately from the insertion portion 91, to be configured to operate the insertion portion 91 by remote control.

The endoscope system 100 having the configuration as described above has the imaging portion 80 equipped with the optical unit 1 described above. Therefore, it is compact, and is capable of changing zoom levels quickly, and is suitable for capturing moving images.

Moreover, according to the endoscope system 100, because the coil 11 is arranged in the fixed portion 2 while the magnet 12 is arranged in the movable portion 3, it is not necessary to move a cable connected to the coil 11. Therefore, there is no risk of disconnection caused by the cable moving in limited space in the distal end portion 91a of the endoscope 90, leading to improved durability.

### Other Embodiments

The embodiments to implement the disclosure have so far been explained, but the disclosure is not to be limited to the embodiments described above. For example, the number of magnets arranged in the movable portion 3 is not limited to three sets as described in the first embodiment, and it may be one set, or multiple sets, such as two sets and four sets to be arranged.

Furthermore, although an example in which three balls (the two first balls 8a and the second ball 8b) are included as the sliders that slide in synchronization with movement of the movable portion 3 has been explained, it is applicable if at least two balls are arranged as the slider, as long as they are positioned between the fixed-portion main body 20 and the movable portion 3, and enable smooth movement of the movable portion 3. Furthermore, other than balls, any object that enables smooth movement of the movable portion 3 in synchronization with the movement of the movable portion 3, such as a roller and a caterpillar, can be adopted.

Moreover, the through hole 20a arranged in the fixed-portion main body 20 is only required to enable assembly of the magnet 12, and it may be formed not piercing through to the outer peripheral side in the radial direction.

Furthermore, in the optical unit 1, arrangement of the coil 11 and the magnet 12 may be reversed. That is, the coil 11 may be arranged in the movable portion 3, and the magnet 12 may be arranged in the fixed portion 2.

As explained above, the optical unit and the endoscope according to the disclosure are useful for moving the movable portion smoothly relative to the fixed portion.

According to the disclosure, an effect that a movable portion can be smoothly moved relative to a fixed portion can be produced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An optical unit comprising:
a fixed portion having a cylindrical shape;
a movable portion holding a lens, the movable portion being arranged movably inside the fixed portion;
a first slider that is arranged between the fixed portion and the movable portion, the first slider being configured to slide with a movement of the movable portion;
a second slider that is arranged between the fixed portion and the movable portion, and at a position different from a position of the first slider, the second slider being configured to slide with the movement of the movable portion;
a driver that includes a coil and a magnet, the driver being configured to move the movable portion in a direction of an optical axis of the lens relative to the fixed portion; and
a biasing part that is arranged outside the movable portion, the biasing part being configured to cause the magnet to generate a biasing force to push the movable portion toward the fixed portion, wherein
the fixed portion includes a first rail portion and a second rail portion guiding a movement of the first slider and the second slider, respectively, and extending in a direction of the optical axis,
the biasing part is arranged at a position facing the second rail portion through the optical axis of the lens,
the first slider abuts on the first rail portion, and is pushed in a direction horizontal to the biasing force, and
the second slider abuts on the second rail portion, and is pushed in a direction perpendicular to the biasing force.

2. The optical unit according to claim 1, wherein
the first slider is arrange in the movable portion at a position at which a rotational moment is generated around the first slider.

3. The optical unit according to claim 1, further comprising
a third slider that is aligned in an optical axis direction of the optical unit relative to the first slider and that is present between the fixed portion and the movable portion, the third slider being configured to slide with the movement of the movable portion.

4. The optical unit according to claim 2, wherein
the first slider, the second slider, and the third slider are arranged at positions at which a center of gravity of the movable portion is located within a triangle formed by connecting centers of gravity of the respective sliders, and a pressing force of a component perpendicular to the biasing force is applied to the second slider.

5. The optical unit according to claim 1, wherein
the fixed portion includes a third rail portion extending in the direction of the optical axis, and the first rail portion, the second rail portion, and the third rail portion are arranged at regular intervals along a circumferential direction of the fixed portion.

6. The optical unit according to claim 5, wherein
the movable portion has a hexagonal shape,
includes a first protruding portion, a second protruding portion, and a third protruding portion that are arranged on an outer surface of the movable portion, and
the first protruding portion, the second protruding portion, and the third protruding portion are arranged on surfaces different from one another, and are not adjacent to one another.

7. The optical unit according to claim 6, wherein
the first protruding portion, the second protruding portion, and the third protruding portion are housed in the first rail portion, the second rail portion, and the third rail portion, respectively.

8. The optical unit according to claim 7, wherein
the first protruding portion includes a first concave portion having a groove shape in which one end in a circumferential direction of the movable portion is open, and a third concave portion aligned with the first concave portion in the direction of the optical axis,
the second protruding portion includes a second concave portion having a groove shape in which one end in the circumferential direction of the movable portion is open, and
the first slider, the second slider, and the third slider are arranged in the first concave portion, the second concave portion, and the third concave portion, respectively.

9. The optical unit according to claim 1, wherein
the first slider, the second slider, and the third slider are arranged such that each side of a triangle formed by connecting centers of gravity of the respective sliders is uniform.

10. The optical unit according to claim 1, wherein
the magnet includes three sets of a first magnet and a second magnet that are arranged on different outer surfaces from one another of the movable portion, and
the first magnet and the second magnet are respectively magnetized in a radial direction of the movable portion,
magnetic polarizations of the first magnet and the second magnet are reversed to each other, and
the three sets of the first magnet and the second magnet are arranged at regular intervals along a circumferential direction of the movable portion.

11. The optical unit according to claim 1, wherein
in the fixed portion, a through hole in which a part of the magnet is housed is formed at a position facing the first rail portion and the second rail portion through a center axis of the fixed portion.

12. The optical unit according to claim 1, wherein
the driver includes a plurality of coils aligned in an optical axis direction of the optical unit, and includes a detector that is arranged between the coils, the detector being configured to output a detection signal to detect a position of the movable portion.

13. An endoscope configured to be inserted into an inside of a subject to observe the inside of the subject, the endoscope comprising:
an optical unit;
an imager configured to convert light guided by the optical unit into an electrical signal; and
a controller configured to control driving of the optical unit, wherein
the optical unit includes:
a fixed portion having a cylindrical shape;
a movable portion holding a lens, the movable portion being arranged movably inside the fixed portion;
a first slider that is arranged between the fixed portion and the movable portion, the first slider being configured to slide with a movement of the movable portion;
a second slider that is arranged between the fixed portion and the movable portion, and at a position different from a position of the first slider, the second slider being configured to slide with the movement of the movable portion;
a driver that includes a coil and a magnet, the driver being configured to move the movable portion in a direction of an optical axis of the lens relative to the fixed portion; and
a biasing part that is arranged outside the movable portion, the biasing part being configured to cause the magnet to generate a biasing force to push the movable portion toward the fixed portion, wherein
the fixed portion includes a first rail portion and a second rail portion guiding a movement of the first slider and the second slider, respectively, and extending in a direction of the optical axis,
the biasing part is arranged at a position facing the second rail portion through the optical axis of the lens,
the first slider abuts on the first rail portion, and is pushed in a direction horizontal to the biasing force, and
the second slider abuts on the second rail portion, and is pushed in a direction perpendicular to the biasing force.

14. The endoscope according to claim 13, wherein
the first slider is arrange in the movable portion at a position at which a rotational moment is generated around the first slider.

15. The endoscope according to claim 13, further comprising
a third slider that is aligned in an optical axis direction of the optical unit relative to the first slider and that is present between the fixed portion and the movable portion, the third slider being configured to slide with the movement of the movable portion.

16. The endoscope according to claim 14, wherein
the first slider, the second slider, and the third slider are arranged at positions at which a center of gravity of the movable portion is located within a triangle formed by connecting centers of gravity of the respective sliders.

17. The endoscope according to claim 13, wherein
the fixed portion includes a third rail portion extending in the direction of the optical axis, and the first rail portion, the second rail portion, and the third rail portion are arranged at regular intervals along a circumferential direction of the fixed portion.

18. The endoscope according to claim 17, wherein
the movable portion has a hexagonal shape,
includes a first protruding portion, a second protruding portion, and a third protruding portion that are arranged on an outer surface of the movable portion, and
the first protruding portion, the second protruding portion, and the third protruding portion are arranged on surfaces different from one another, and are not adjacent to one another.

19. The endoscope according to claim 18, wherein
the first protruding portion, the second protruding portion, and the third protruding portion are housed in the first rail portion, the second rail portion, and the third rail portion, respectively.

20. The endoscope according to claim 19, wherein
the first protruding portion includes a first concave portion having a groove shape in which one end in a circumferential direction of the movable portion is open, and a third concave portion aligned with the first concave portion in the direction of the optical axis,
the second protruding portion includes a second concave portion having a groove shape in which one end in the circumferential direction of the movable portion is open, and
the first slider, the second slider, and the third slider are arranged in the first concave portion, the second concave portion, and the third concave portion, respectively.
